(19) United States
(12) Patent Application Publication
(10) Pub. No.: US 2026/0345496 A1
(43) Pub. Date: Nov. 5, 2026

(54) Tafamidis Pharmaceutical Compositions

(71) Applicant: Pfizer Inc., New York, NY (US)

(72) Inventor: John Edward Kresevic, Richmond, RI (US)

(73) Assignee: Pfizer Inc., New York, NY (US)

(21) Appl. No.: 19/099,295

(22) PCT Filed: Jul. 25, 2023
(86) PCT No.: PCT/IB2023/057546

§ 371 Date: Jan. 28, 2025

Related U.S. Application Data
(60) Provisional application No. 63/369,696, filed on Jul. 28, 2022.
(60) Provisional application No. 63/514,038, filed on Jul. 17, 2023.

(51) Int. Cl. A61K 9/20; A61K 9/28; A61K 31/423; A61K 47/12

(52) U.S. Cl. CPC A61K 9/2027; A61K 9/2018; A61K 9/2095; A61K 9/2866; A61K 9/2893; A61K 31/423; A61K 47/12

(57) ABSTRACT

The present invention provides a pharmaceutical composition which is a tablet comprising tafamidis free acid, one or more diluents, 7-9% (w/w %) disintegrant and a lubricant. A representative example is a tablet comprising about 11.6% (w/w %) tafamidis free acid; about 53.1% (w/w %) microcrystalline cellulose; about 26.55% (w/w %) lactose monohydrate; about 8.0% (w/w %) crospovidone type b and about 0.75% (w/w %) magnesium stearate.

Transthyretin (ATTR) amyloidosis is rare, progressive disease characterized by the abnormal buildup of amyloid deposits composed of misfolded transthyretin protein in the body's organs and tissues. ATTR amyloidosis can impact numerous organs and tissues in the body, including the peripheral nervous system, and organs such as the heart, kidneys, gastrointestinal tract and eyes. Transthyretin cardiomyopathy (ATTR-CM) and transthyretin polyneuropathy (ATTR-PN) are two presentations of the disease. ATTR-CM affects the heart and leads to restrictive cardiomyopathy and progressive heart failure. There are two sub-types of ATTR-CM: hereditary, which is caused by a mutation in the transthyretin gene and can occur in people as early as their 50s and 60s; or the wild-type form which is associated with aging, and is thought to be more common, usually affecting men after age 60. Often ATTR-CM is diagnosed only after symptoms have become severe. ATTR-PN results from a genetic mutation of the transthyretin gene causing amyloid fibrils to form in the peripheral and autonomic nerves. ATTR-PN typically occurs during active adult years with onset as early as the 30s in some patients, followed by disease progression that may reach the terminal stage in approximately 10 years on average from disease onset.

Tafamidis, sold under the brand names Vyndaqel® and Vyndamax®, is an oral transthyretin stabilizer medication used for the treatment of adults with certain forms of transthyretin amyloidosis. Tafamidis can be used to treat both hereditary forms as well as wild-type transthyretin amyloidosis. Tafamidis works by selectively binding to transthyretin and stabilizes the quaternary structure of the tetrameric transthyretin protein and slowing the formation of amyloid. Tafamidis free acid can be prepared by methods such as those described in U.S. Pat. No. 7,214,695, WO 04056315, U.S. Pat. No. 9,770,441 and WO 2016/038500.

### SUMMARY OF THE INVENTION

The present invention provides oral pharmaceutical compositions comprising tafamidis free acid. The oral pharmaceutical compositions comprise tafamidis free acid in an oral tablet dosage form. The present invention particularly provides immediate release tablet formulations of tafamidis free acid wherein the tablet may be film-coated. Tafamidis is formulated as a common blend to make immediate release tablet cores, which can then be film-coated to make film-coated tablets, such as 12.2 mg tafamidis free acid and 61 mg tafamidis free acid tablet drug products.

The tafamidis 12.2 mg and 61 mg film-coated tablets have been formulated for immediate release for the oral treatment of transthyretin amyloidosis diseases such as ATTR-CM and ATTR-PN. The tablet cores are made from a common blend using a dry granulation process and can then be film-coated by a batch process to provide the film-coated tablet drug product.

The following are representative embodiments of the present invention and are not to be construed in a limiting manner.
E1 is a pharmaceutical composition which is a tablet comprising tafamidis free acid, one or more diluents, 7-9% (w/w %) disintegrant and a lubricant.
E2 is the pharmaceutical composition of E1 which is a tablet wherein the disintegrant is crospovidone.
E3 is the pharmaceutical composition of E1 or E2 which is a tablet wherein the disintegrant is crospovidone type b.
E4 is the pharmaceutical composition of any one of E1 to E3 which is a tablet wherein the one or more diluents are selected from microcrystalline cellulose and lactose monohydrate.
E5 is the pharmaceutical composition of any one of E1 to E4 which is a tablet wherein the lubricant is magnesium stearate.
E6 is the pharmaceutical composition of any one of E1 to E5 which is a tablet comprising about 8% (w/w %) crospovidone type b.
E7 is the pharmaceutical composition of any one of E1 to E6 which is a tablet comprising about 11.6% (w/w %) tafamidis free acid.
E8 is the pharmaceutical composition of any one of E1 to E7 which is a tablet comprising 12.2 mg tafamidis free acid.
E9 is the pharmaceutical composition of any one of E1 to E7 which is a tablet comprising 61 mg tafamidis free acid.
E10 is the pharmaceutical composition of any one of E1 to E9 which is a tablet comprising about 79.65% (w/w %) of one or more diluents which are selected from microcrystalline cellulose and lactose monohydrate.
E11 is the pharmaceutical composition of any one of E1 to E10 which is a tablet comprising about 53.1% (w/w %) microcrystalline cellulose and about 26.55% lactose monohydrate.
E12 is the pharmaceutical composition of any one of E1 to E11 which is a tablet comprising about 0.75% (w/w %) of magnesium stearate.
E13 is the pharmaceutical composition of any one of E1 to E12 wherein the tablet is film coated.
E14 is the pharmaceutical composition of E13 wherein the tablet film coating comprises hydroxypropyl methylcellulose and lactose.
E15 is the pharmaceutical composition of E13 or E14 wherein the tablet film coating is about 4% (w/w) of the overall weight of the film coated tablet.
E16 is a pharmaceutical composition which is a tablet comprising about 11.6% (w/w %) tafamidis free acid; about 53.1% (w/w %) microcrystalline cellulose; about 26.55% (w/w %) lactose monohydrate; about 8.0% (w/w %) crospovidone type b and about 0.75% (w/w %) magnesium stearate.
E17 is a pharmaceutical composition which is a tablet comprising 11.6%±0.1% (w/w %) tafamidis free acid; 53.1%±0.1% (w/w %) microcrystalline cellulose; 26.55%±0.1% (w/w %) lactose monohydrate; 8.0%±0.1% (w/w %) crospovidone type b and 0.75%±0.1% (w/w %) magnesium stearate.
E18 is the pharmaceutical composition of E16 or E17 wherein the tablet is film coated.
E19 is the pharmaceutical composition of any one of E16 to E18 wherein the tablet film coating comprises hydroxypropyl methylcellulose and lactose.
E20 is the pharmaceutical composition of E18 or E19 wherein the tablet film coating is 4.0%±0.1% (w/w %) of the overall weight of the film coated tablet.
E21 is a pharmaceutical composition which is a tablet comprising about 12.20 mg tafamidis free acid, about 55.85 mg microcrystalline cellulose, about 27.92 mg lactose monohydrate, about 8.42 mg crospovidone type b and about 0.79 mg magnesium stearate.
E22 is the pharmaceutical of E21 which is a tablet comprising 12.20 mg tafamidis free acid, 55.85 mg microcrystalline cellulose, 27.92 mg lactose monohydrate, 8.42 mg crospovidone type b and 0.79 mg magnesium stearate.
E23 is the pharmaceutical composition of E21 or E22 wherein the tablet is film coated.
E24 is the pharmaceutical composition of E23 wherein tablet film coating comprises hydroxypropyl methylcellulose and lactose.
E25 is the pharmaceutical composition of E23 or E24 wherein the tablet film coating weighs 4.21 mg.
E26 is a pharmaceutical composition which is a tablet comprising about 61.00 mg tafamidis free acid, about 279.25 mg microcrystalline cellulose, about 139.6 mg lactose monohydrate, about 42.10 mg crospovidone type b and about 2.95 mg magnesium stearate.
E27 is the pharmaceutical composition of E26 which is a tablet comprising 61.00 mg tafamidis free acid, 279.25 mg microcrystalline cellulose, 139.6 mg lactose monohydrate, 42.10 mg crospovidone type b and 2.95 mg magnesium stearate.
E28 is the pharmaceutical composition of E26 or E27 wherein the tablet is film coated.
E29 is the pharmaceutical composition of E28 wherein the tablet film coating comprises hydroxypropyl methylcellulose and lactose.
E30 is the pharmaceutical composition of E28 or E29 wherein the tablet film coating weighs 21.05 mg.
E31 is the pharmaceutical composition of any one of E1 to E7 which is a tablet comprising about 13.0 mg, about 13.5 mg, about 14.0 mg, about 14.5 mg, about 15.0 mg, about 15.5 mg, about 59.0 mg, about 60.0 mg or about 62 mg tafamidis free acid.
E32 is the pharmaceutical composition of E31 which is a tablet comprising 13.0 mg, 13.5 mg, 14.0 mg, 14.5 mg, 15.0 mg, 15.5 mg, 59.0 mg, 60.0 mg or 62.0 mg of tafamidis free acid.
E33 is the pharmaceutical composition of E32 which is a tablet comprising 13.0 mg tafamidis free acid.
E34 is the pharmaceutical composition of E33 which is a tablet comprising 13.0 mg tafamidis free acid, 59.51 mg microcrystalline cellulose, 29.75 mg lactose monohydrate, 8.98 mg crospovidone type b and 0.85 mg magnesium stearate.
E35 is the pharmaceutical composition of E32 which is a tablet comprising 13.5 mg tafamidis free acid.
E36 is the pharmaceutical composition of E35 which is a tablet comprising 13.5 mg tafamidis free acid, 61.80 mg microcrystalline cellulose, 30.89 mg lactose monohydrate, 9.32 mg crospovidone type b and 0.88 mg magnesium stearate.
E37 is the pharmaceutical composition of E32 which is a tablet comprising 14.0 mg tafamidis free acid.
E38 is the pharmaceutical composition of E37 which is a tablet comprising 14.0 mg tafamidis free acid, 64.09 mg microcrystalline cellulose, 32.04 mg lactose monohydrate, 9.66 mg crospovidone type b and 0.91 mg magnesium stearate.
E39 is the pharmaceutical composition of E32 which is a tablet comprising 14.5 mg tafamidis free acid.
E40 is the pharmaceutical composition of E39 which is a tablet comprising 14.5 mg tafamidis free acid, 66.38 mg microcrystalline cellulose, 33.18 mg lactose monohydrate, 10.00 mg crospovidone type b and 0.94 mg magnesium stearate.
E41 is the pharmaceutical composition of E32 which is a tablet comprising 15.0 mg tafamidis free acid.
E42 is the pharmaceutical composition of E41 which is a tablet comprising 15.0 mg tafamidis free acid, 68.69 mg microcrystalline cellulose, 34.33 mg lactose monohydrate, 10.36 mg crospovidone type b and 0.97 mg magnesium stearate.
E43 is the pharmaceutical composition of E32 which is a tablet comprising 15.5 mg tafamidis free acid.
E44 is the pharmaceutical composition of E43 which is a tablet comprising 15.5 mg tafamidis free acid, 70.96 mg microcrystalline cellulose, 35.47 mg lactose monohydrate, 10.70 mg crospovidone type b and 1.00 mg magnesium stearate.
E45 is the pharmaceutical composition of E32 which is a tablet comprising 59.0 mg tafamidis free acid.
E46 is the pharmaceutical composition of E45 which is a tablet comprising 59.0 mg tafamidis free acid, 270.09 mg microcrystalline cellulose, 135.02 mg lactose monohydrate, 40.72 mg crospovidone type b and 3.82 mg magnesium stearate.
E47 is the pharmaceutical composition of E32 which is a tablet comprising 60.0 mg tafamidis free acid.
E48 is the pharmaceutical composition of E47 which is a tablet comprising 60.0 mg tafamidis free acid, 274.67 mg microcrystalline cellulose, 137.31 mg lactose monohydrate, 41.40 mg crospovidone type b and 3.89 mg magnesium stearate.
E49 is the pharmaceutical composition of E32 which is a tablet comprising 62.0 mg tafamidis free acid.
E50 is the pharmaceutical composition of E49 which is a tablet comprising 62.0 mg tafamidis free acid, 283.83 mg microcrystalline cellulose, 141.89 mg lactose monohydrate, 42.80 mg crospovidone type b and 4.01 mg magnesium stearate.
E51 is the pharmaceutical composition of any one of E31 to E50 wherein the tablet is film coated.
E52 is the pharmaceutical composition of E51 wherein the tablet film coating comprises hydroxypropyl methylcellulose and lactose.
E53 is the pharmaceutical composition of E52 wherein the tablet film coating is about 4% (w/w) of the overall weight of the film coated tablet.
E54 is a method of treating transthyretin amyloidosis in a patient comprising administering a pharmaceutical composition according to any one of E1 to E53 to the patient.
E55 is the method of claim 54 wherein the transthyretin amyloidosis is transthyretin cardiomyopathy (ATTR-CM) or transthyretin polyneuropathy (ATTR-PN).
E56 is the use of a pharmaceutical composition according to any one of E1 to E53 for treatment of transthyretin amyloidosis.
E57 is the use according to E56 wherein the transthyretin amyloidosis is transthyretin cardiomyopathy (ATTR-CM) or transthyretin polyneuropathy (ATTR-PN).

### BRIEF DESCRIPTION OF THE DRAWINGS

FIG. 1: Flow Diagram of Manufacturing Process for Tafamidis 12.2 mg and 61.0 mg Film-Coated Tablets.

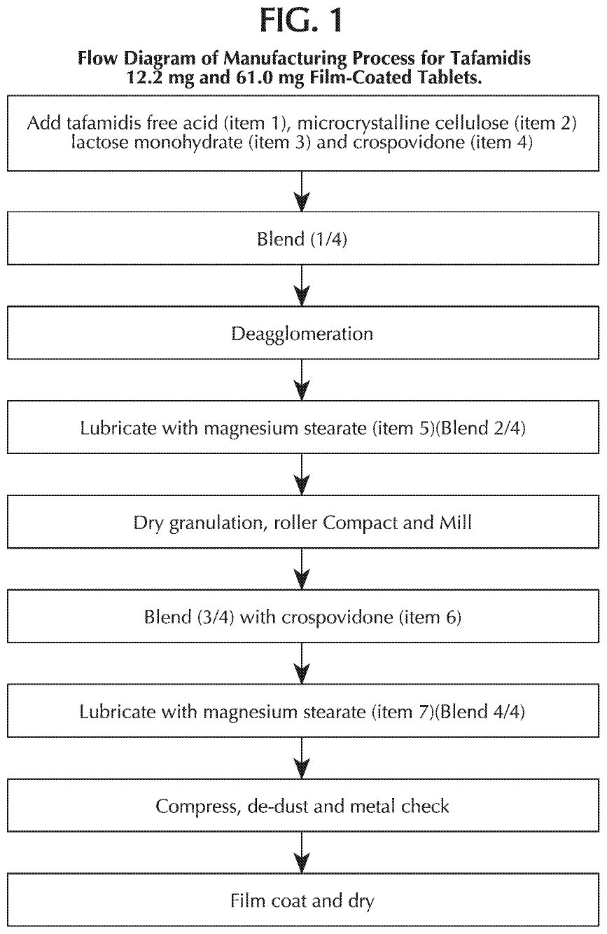

### DETAILED DESCRIPTION OF THE INVENTION

The excipients used in the tafamidis tablet or film-coated tablet are globally acceptable and all but crospovidone are present at precedented levels in the formulation. The amount of tablet ingredients can be expressed as weight percent of the tablet (i.e. w/w %). It is to be used that the term about can be plus or minus 0.2% or alternatively 0.1% (i.e. ±0.2% or ±0.1%). Crospovidone is typically used at a 1-5% (w/w %) level and an advantageous embodiment of the tafamidis common blend used to prepare the tablet core of the present invention contains 8% crospovidone. Crospovidone was evaluated at 4, 6, 8 and 10% (w/w %) levels during development and the microcrystalline cellulose/lactose monohydrate diluents were adjusted accordingly to manage the tablet core weight. Optimal release characteristics to achieve bioequivalence (BE) when compared to currently marketed tafamidis meglumine 20 mg soft gelatin capsules product based on biopharmaceutical modeling coupled with the use of a discriminatory dissolution methodology (in-vitro) was achieved at the 8% (w/w %) crospovidone level. An alternate formulation evaluated at a reduced 6% (w/w %) crospovidone disintegrant level as provided in Table 2, failed bioequivalence when compared to tafamidis meglumine 20 mg soft gelatin capsules during a batch evaluation. This data indicates the criticality of the crospovidone disintegrant level required to achieve bioequivalence to the 20 mg tafamidis meglumine gel capsule.

Alterations of the formulation to achieve bioequivalence (BE) as compared to the alternate formulations include increased disintegrant level, change in crospovidone particle size and elimination of the dry binder (vinylpyrrolidone-vinyl acetate copolymers or commonly known as copovidone). It is hypothesized that the use of the reduced particle size of crospovidone for the formulation containing crospovidone in the extra-granular portion of the manufacturing process acts as a disintegrant/dry binder thus eliminating the drug substance hold up within the tablet in-vivo as potentially the root cause to alternate formulation BE failure as noted with vinylpyrrolidone-vinyl acetate copolymers (dry binder) with no disintegration properties. Studies have been performed to evaluate the impact of excipients on drug product manufacturing and performance. Microcrystalline cellulose and lactose monohydrate are diluents used in the present tafamidis formulation and have precedents in oral tablets at the proposed levels. These ingredients are used in a 2:1 ratio to promote powder flow and compactability during tablet compression. Crospovidone is included in the tablet core formulation as a disintegrant in the oral tablets. Adding the disintegrant promotes tablet disintegration and adding in the extra-granular portion after roller compaction is completed acts as a disintegrant as well as a dry binder. Magnesium stearate is included in the tablet core formulation as a lubricant in the tablets. Magnesium stearate is used as a lubricant in the tablet core formulation to aid in tablet compression. Opadry® II yellow and Opadry® II white are proprietary film coating systems used for the 12.2 mg and 61 mg tablets, respectively.

### Dose Adjustment Estimate for Low Dose Tafamidis Free Acid Tablet

To estimate the potential dose adjustment range for low dose tafamidis free acid (FA) tablet (equivalent to one 20 mg meglumine salt (MS) capsule), the Cₘₐₓ and AUC_{inf} ratio of 12.2 mg FA tablet to 20 mg tafamidis MS capsule obtained in Study B3461103 was used. Cₘₐₓ and AUC_{inf} of a tafamidis FA table are assumed to increase proportionally as the dose increases from 12.2 mg to 17 mg. The estimated Cₘₐₓ and AUC_{inf} ratios of a tafamidis FA tablet with various strengths to the 20 mg tafamidis MS capsule are listed in Table A.

TABLE A
Estimated Cₘₐₓ and AUC_{inf} ratio of dose-adjusted tafamidis
free acid tablet (test) to 20 mg meglumine capsule (reference)
calculated by clinical data observed in B3461103.

| | Cₘₐₓ ratio | AUC_{inf} ratio |
|---|---|---|
| Dose (mg) | (FA Tab/MS Cap) | (FA Tab/MS Cap) |
| 12 | 79.67 | 91.65 |
| 12.2 (observed) | 81.00 | 93.18 |
| 13 | 86.31 | 99.29 |
| 14 | 92.95 | 106.39 |
| 15 | 99.59 | 114.57 |
| 15.5 | 102.91 | 118.38 |
| 16 | 106.23 | 122.20 |
| 17 | 112.87 | 129.84 |

The potential dose adjustment range for low dose tafamidis FA tablet (equivalent to 20 mg tafamidis MS capsule) is estimated to be 12.2 mg to 16 mg as the estimated Cₘₐₓ and AUC_{inf} ratios are within 0.8-1.25. A more conservative estimate would set the dose range between 13 mg to 15.5 mg as the estimated Cₘₐₓ and AUC_{inf} ratios would be within 0.85-1.20.

### Dose Adjustment Estimate for High Dose Tafamidis Free Acid Tablet

To estimate the potential dose adjustment range for high dose tafamidis FA tablet (equivalent to 4×20 mg meglumine salt capsules), the Cₘₐₓ and AUC_{inf} ratios of a 48.8 mg or 58 mg tafamidis FA tablet to 4×20 mg tafamidis MS capsules obtained in Study B3461030 and Study B3461051 were used. Cₘₐₓ and AUC_{inf} of a tafamidis FA table are assumed to increase proportionally as the dose increases from 48.8 mg to 71 mg.

The estimated Cₘₐₓ and AUC_{inf} ratios can be calculated using the clinical data obtained in Study B3461030 (48.8 mg tafamidis FA tablet vs 4×20 mg tafamidis MS capsules) and Study B3461051 (48.8 mg tafamidis FA tablet or 58 mg tafamidis free acid tablet vs. 4×20 mg tafamidis MS capsules). The estimated Cₘₐₓ and AUC_{inf} ratios of tafamidis FA tablet with various strengths to 4×20 mg tafamidis MS capsules are listed in Table B.

Based on the observed rBA data of the 48.8 mg tafamidis FA tablet in Study B3461030 (Table B2), the dose adjustment range for high dose tafamidis FA tablet (equivalent to 4×20 mg meglumine salt capsule) is estimated to be 51 mg to 70 mg as the estimated Cₘₐₓ and AUC_{inf} ratios are within 0.8-1.25. With the same acceptance criteria, the dose adjustment range is estimated to be 53 mg to 67 mg using the observed rBA data of the 48.8 mg tafamidis FA tablet in Study B3461051 (Table B2), and 56 mg to 65 mg using the observed rBA data of the 58 mg tafamidis FA tablet in Study B3461051 (Table B3).

To estimate the dose adjustment range with a more conservative approach, the acceptance criteria of Cₘₐₓ and AUC_{inf} ratios can be set at 0.85-1.20. In this case, the potential dose adjustment range is estimated to be 51-67 mg using the 48.8 mg tafamidis FA tablet rBA data from Study B3461030 (Table B), 56-64 mg using the 48.8 mg tafamidis FA tablet rBA data from Study B3461051 (Table B2), and 59-62 mg using the 58 mg tafamidis FA tablet rBA data from study B3461051 (Table B3). As all the calculated ranges cover 59-62 mg, this dose adjustment range (59-62 mg) could be preferable.

In summary, the observed clinical data suggests that the dose adjustment range of high dose tafamidis FA tablet is likely between 51 mg to 70 mg. This dose range include all the estimated ranges calculated by the methods described above. Within this dose adjustment range, 59 mg to 62 mg is preferable because it is the intersection of all the estimated ranges.

Table B. Estimated Cₘₐₓ and AUC_{inf} ratio between dose-adjusted tafamidis free acid tablet (test) and 4×20 mg meglumine capsules (reference).

Cₘₐₓ and AUC_{inf} ratio calculated using rBA data between 48.8 mg tafamidis free acid tablet and 4×20 mg tafamidis meglumine salt capsules in study 1030.

TABLE B
Estimated Cₘₐₓ and AUC_{inf} ratio between
dose-adjusted tafamidis free acid tablet (test) and 4 × 20 mg meglumine
capsules (reference). Cₘₐₓ and AUC_{inf} ratio
calculated using rBA data between 48.8 mg tafamidis free acid tablet and
4 × 20 mg tafamidis meglumine salt capsules in study 1030.

| | Cₘₐₓ ratio | AUC_{inf} ratio |
|---|---|---|
| Dose (mg) | (FA Tab/MS Cap) | (FA Tab/MS Cap) |
| 48.8 (observed) | 79.67 | 89.00 |
| 50 | 78.46 | 90.78 |
| 51 | 80.03 | 92.56 |
| 52 | 81.60 | 94.34 |
| 53 | 83.17 | 96.12 |
| 54 | 84.74 | 97.90 |
| 55 | 86.31 | 99.68 |
| 60 | 94.16 | 108.58 |
| 61 | 95.73 | 108.58 |
| 62 | 97.29 | 110.35 |
| 63 | 98.86 | 112.13 |
| 64 | 100.43 | 113.91 |
| 65 | 102.00 | 115.69 |
| 66 | 103.57 | 117.47 |
| 67 | 105.14 | 119.25 |
| 68 | 106.71 | 121.03 |
| 69 | 108.28 | 122.81 |
| 70 | 109.85 | 124.59 |
| 71 | 111.42 | 126.37 |

Table B2: Cₘₐₓ and AUC_{inf} ratio calculated using rBA data between 48.8 mg tafamidis free acid tablet and 4×20 mg tafamidis meglumine salt capsules in study 1051

TABLE B2
Cₘₐₓ and AUC_{inf} ratio calculated using rBA data between
48.8 mg tafamidis free acid tablet and 4 × 20
mg tafamidis meglumine salt capsules in study 1051

| | Cₘₐₓ ratio | AUC_{inf} ratio |
|---|---|---|
| Dose (mg) | (FA Tab/MS Cap) | (FA Tab/MS Cap) |
| 48.8 (observed) | 74.48 | 90.93 |
| 50 | 76.31 | 93.17 |
| 51 | 77.84 | 95.03 |
| 52 | 79.36 | 96.89 |
| 53 | 80.89 | 98.76 |
| 54 | 82.42 | 100.62 |
| 55 | 83.94 | 102.48 |
| 56 | 85.47 | 104.35 |
| 57 | 87.00 | 106.21 |
| 58 | 88.52 | 108.07 |
| 59 | 90.05 | 109.94 |
| 60 | 91.57 | 111.80 |
| 61 | 93.10 | 113.66 |
| 62 | 94.63 | 115.53 |
| 63 | 96.15 | 117.39 |
| 64 | 97.68 | 119.25 |
| 65 | 99.20 | 121.12 |
| 66 | 100.73 | 122.98 |
| 67 | 102.26 | 124.84 |
| 68 | 103.78 | 126.71 |
| 69 | 105.31 | 128.57 |

Table B3: Cₘₐₓ and AUC_{inf} ratio calculated using rBA data between 58 mg tafamidis free acid tablet and 4×20 mg tafamidis meglumine salt capsules in study 1051

TABLE B3
Cₘₐₓ and AUC_{inf} ratio calculated using rBA data between
58 mg tafamidis free acid tablet and 4 × 20
mg tafamidis meglumine salt capsules in study 1051

| | Cₘₐₓ ratio | AUC_{inf} ratio |
|---|---|---|
| Dose (mg) | (FA Tab/MS Cap) | (FA Tab/MS Cap) |
| 58 (observed) | 83.68 | 111.15 |
| 55 | 79.35 | 105.40 |
| 56 | 80.79 | 107.32 |
| 57 | 82.24 | 109.23 |
| 58 | 83.68 | 111.15 |
| 59 | 85.12 | 113.07 |
| 60 | 86.57 | 114.98 |
| 61 | 88.01 | 116.90 |
| 62 | 89.45 | 118.82 |
| 63 | 90.89 | 120.73 |
| 64 | 92.34 | 122.65 |
| 65 | 93.78 | 124.56 |
| 66 | 95.22 | 126.48 |

### Formulation

Tafamidis is formulated as a common blend to make immediate release tablet cores such as 12.2 mg and 61 mg immediate release tablet cores. The formulation, provided in Table 1, is composed of microcrystalline cellulose and lactose monohydrate (diluents), crospovidone (disintegrant), and magnesium stearate (lubricant). Commercial tafamidis tablet cores are manufactured using a dry granulation manufacturing platform followed by a film coating step. Tablets are film coated with an appropriate coating. For example the tablets can be coated with hydroxypropyl methylcellulose/lactose based Opadry® II formulation with yellow color for the 12.2 mg tablets and white color for the 61 mg tablets.

### EXAMPLES

### General Procedure for Tablet Preparation

Add the microcrystalline cellulose (item 2), intra-granular crospovidone (item 4), tafamidis free acid (item 1), and lactose monohydrate (item 3) into an appropriately sized Intermediate Bulk Container (IBC) and blend for 20±1 minutes at 12±1 revolutions per minute (rpm) to provide a first blend [designated Blend ¼].

Pass the contents of the IBC (Blend ¼) through a Comil U20 fitted with a 032R or 024R screen and round edge impeller operated at 750±50 rpm, or equivalent (e.g. Comil U10/197 S equipped with a 0.6-0.8 mm sieve operated at 1320±80 rpm). Collect the resulting blend in an IBC. Pre-screen (1 mm screen, US Sieve #20) the intra-granular magnesium stearate (Item 5) and add to the IBC. Blend for 3±1 minutes at 12±1 rpm to provide a second blend [designated Blend 2/4]. Roller compact and mill the Blend 2/4 on a Gerteis 3W Macropactor or equivalent. Collect the resulting granules in an IBC.

Add extra-granular crospovidone (Item 6), adjusting for yield if required, into the IBC and blend for 20±1 minutes. Pre-screen (1 mm screen, US Sieve #20) extra-granular magnesium stearate (Item 7) and add to the IBC, adjusting for yield if required. Blend for 3±1 minutes at 12±1 rpm [Blend 4/4] ±1 minutes at 12±1 rpm [designated Blend 3/4].

Using a Rotary Press compress tablets using 6.5 mm tooling to a target average weight of 105.2±5% (±5.2 mg) and target hardness of 5±2 kP (49±19 N). The preceding sentence is for the 12.2 mg tablets and an analogous tablet compression process is used to prepare the 61 mg tablets. Following compression, de-dust the cores and pass them through a metal detector.

For the 12.2 mg tablets prepare an 18% solids w/w film coating suspension using Opadry II Yellow (33G120011) (Item 8) and purified water (Item 9). This suspension should be prepared in excess to account for processing loss. An analogous film coating suspension is prepared for the 61 mg tablets using Opadry II White and purified water. Fill a suitable coating pan with the appropriate amount of tablet cores. Apply sufficient film coating to a minimum target weight gain of 4% based on the average core tablet weight.

The preceding procedure is used in an analogous manner to prepare the tablet cores which contain 13.0 mg, 13.5 mg, 14.0 mg, 14.5 mg, 15.0 mg, 15.5 mg, 59.0 mg, 60 mg or 62.0 mg of tafamidis and to coat those cores with 4% by weight of film coating.

Examples 1 and 2 were prepared using the General Procedure for Tablet Preparation as described above.

Table 1: Examples 1 and 2-Composition of Tafamidis 12.2 mg and 61 mg Film-Coated Tablets using 8% (w/w) Disintegrant Crospovidone Type B

TABLE 1
Examples 1 and 2 - Composition of Tafamidis 12.2 mg and 61 mg Film-
Coated Tablets using 8% (w/w) Disintegrant Crospovidone Type B

| | | 12.2 mg tafamidis | 61 mg tafamidis |
|---|---|---|---|
| | | tablet | tablet |
| | | (Example 1) | (Example 2) |
| Ingredient | Function | (mg/tablet) | (mg/tablet) |
| Tafamidis (free acid) | Active Ingredient | 12.20 | 61.00 |
| Microcrystalline | Diluent | 55.85 | 279.25 |
| cellulose | | | |
| Lactose Monohydrate | Diluent | 27.92 | 139.6 |
| Crospovidone Type B | Disintegrant | 4.21 | 21.05 |
| (super fine) ^{a} | | | |
| Magnesium stearate ^{a c} | Lubricant | 0.53 | 2.65 |
| Crospovidone Type B | Disintegrant | 4.21 | 21.05 |
| (super fine) ^{b} | | | |
| Magnesium stearate ^{b c} | Lubricant | 0.26 | 1.30 |
| Total Tablet Core Weight | | 105.18 | 525.90 |
| Opadry ® II yellow | Film-coating agent | 4.21 | — |
| Opadry ® II white | Film-coating agent | — | 21.05 |

| | | |
|---|---|---|
| Total Film-Coated Tablet Weight | 109.39 | 546.95 |

^{a} Intra-granular
^{b} Extra-granular
^{c} non-bovine

### Comparator Example A

Table 2: Comparator Example A-Composition of Tafamidis 12.2 mg Film-Coated Tablet using 6% (w/w) Disintegrant Crospovidone Type A

TABLE 2
Comparator Example A - Composition of Tafamidis 12.2 mg Film-
Coated Tablet using 6% (w/w) Disintegrant Crospovidone Type A

| | | 12.2 mg tafamidis |
|---|---|---|
| | | tablet |
| Ingredient | Function | (mg/tablet) |
| Tafamidis (free acid) | Active Ingredient | 12.20 |
| Microcrystalline | Diluent | 55.14 |
| cellulose | | |
| Lactose Monohydrate | Diluent | 27.58 |
| Crospovidone Type A ^{a} | Disintegrant | 2.10 |
| Magnesium stearate ^{a c} | Lubricant | 0.53 |
| Crospovidone Type A ^{b} | Disintegrant | 4.21 |
| Copovidone | Dry Binder | 3.16 |
| Magnesium stearate ^{b c} | Lubricant | 0.26 |
| Total Tablet Core Weight | | 105.18 |
| Opadry ® II yellow | Film-coating agent | 4.21 |
| Opadry ® II white | Film-coating agent | — |

| | |
|---|---|
| Total Film-Coated Tablet Weight | 109.39 |

^{a} Intra-granular
^{b} Extra-granular
^{c} non-bovine

The above tablet was prepared in a manner analogous to the General Method described above for Examples 1 and 2. In studies this formulation failed to exhibit equivalent properties to the 20 mg tafamidis meglumine gel capsule and therefore was determined to be not viable as a commercially acceptable formulation.

### Comparator Example B

Table 3: Composition of Tafamidis 12.2 mg Film-Coated Tablet using 4% (w/w) Disintegrant Crospovidone Type A

TABLE 3
Composition of Tafamidis 12.2 mg Film-Coated Tablet
using 4% (w/w) Disintegrant Crospovidone Type A

| | | 12.2 mg tafamidis |
|---|---|---|
| | | tablet |
| Ingredient | Function | (mg/tablet) |
| Tafamidis (free acid) | Active Ingredient | 12.20 |
| Microcrystalline | Diluent | 55.14 |
| cellulose | | |
| Lactose Monohydrate | Diluent | 27.58 |
| Crospovidone Type A ^{a} | Disintegrant | 2.10 |
| Magnesium stearate ^{a c} | Lubricant | 0.53 |
| Crospovidone Type A ^{b} | Disintegrant | 4.21 |
| Copovidone | Dry Binder | 3.16 |
| Magnesium stearate b c | Lubricant | 0.26 |
| Total Tablet Core Weight | | 105.18 |
| Opadry ® II yellow | Film-coating agent | 4.21 |
| Opadry ® II white | Film-coating agent | — |

| | |
|---|---|
| Total Film-Coated Tablet Weight | 109.39 |

^{a} Intra-granular
^{b} Extra-granular
^{c} non-bovine

### Examples 3-12

Table 1A: Examples 3 and 4-Composition of Tafamidis 13.0 mg and 13.5 mg Film-Coated Tablets using 8% (w/w) Disintegrant Crospovidone Type B

TABLE 1A
Examples 3 and 4 - Composition of Tafamidis 13.0 mg and 13.5 mg Film-
Coated Tablets using 8% (w/w) Disintegrant Crospovidone Type B

| | | 13.0 mg tafamidis | 13.5 mg |
|---|---|---|---|
| | | tablet | tafamidis tablet |
| | | (Example 3) | (Example 4) |
| Ingredient | Function | (mg/tablet) | (mg/tablet) |
| Tafamidis (free acid) | Active Ingredient | 13.00 | 13.50 |
| Microcrystalline | Diluent | 59.51 | 61.80 |
| cellulose | | | |
| Lactose Monohydrate | Diluent | 29.75 | 30.89 |
| Crospovidone Type B | Disintegrant | 4.49 | 4.66 |
| (super fine) ^{a} | | | |
| Magnesium stearate ^{a c} | Lubricant | 0.57 | 0.59 |
| Crospovidone Type B | Disintegrant | 4.49 | 4.66 |
| (super fine) ^{b} | | | |
| Magnesium stearate ^{b c} | Lubricant | 0.28 | 0.29 |
| Total Tablet Core Weight | | 112.09 | 116.39 |
| Opadry ® II yellow | Film-coating agent | 4.48 | 4.66 |
| Opadry ® II white | Film-coating agent | — | |

| | | |
|---|---|---|
| Total Film-Coated Tablet Weight | 116.57 | 121.05 |

^{a} Intra-granular
^{b} Extra-granular
^{c} non-bovine

Table 1B: Examples 5 and 6-Composition of Tafamidis 14.0 mg and 14.5 mg Film-Coated Tablets using 8% (w/w) Disintegrant Crospovidone Type B

TABLE 1B
Examples 5 and 6 - Composition of Tafamidis 14.0 mg and 14.5 mg Film-
Coated Tablets using 8% (w/w) Disintegrant Crospovidone Type B

| | | 14.0 mg tafamidis | 14.5 mg |
|---|---|---|---|
| | | tablet | tafamidis tablet |
| | | (Example 5) | (Example 6) |
| Ingredient | Function | (mg/tablet) | (mg/tablet) |
| Tafamidis (free acid) | Active Ingredient | 14.00 | 14.50 |
| Microcrystalline | Diluent | 64.09 | 66.38 |
| cellulose | | | |
| Lactose Monohydrate | Diluent | 32.04 | 33.18 |
| Crospovidone Type B | Disintegrant | 4.83 | 5.00 |
| (super fine) ^{a} | | | |
| Magnesium stearate ^{a c} | Lubricant | 0.61 | 0.63 |
| Crospovidone Type B | Disintegrant | 4.83 | 5.00 |
| (super fine) ^{b} | | | |
| Magnesium stearate ^{b c} | Lubricant | 0.30 | 0.31 |
| Total Tablet Core Weight | | 120.70 | 125.00 |
| Opadry ® II yellow | Film-coating agent | 4.83 | 5.00 |
| Opadry ® II white | Film-coating agent | — | — |

| | | |
|---|---|---|
| Total Film-Coated Tablet Weight | 125.53 | 130.00 |

^{a} Intra-granular
^{b} Extra-granular
^{c} non-bovine

TABLE 1C
Examples 7 and 8-Composition of Tafamidis 15.0 mg
and 15.5 mg Film-Coated Tablets using 8%
(w/w) Disintegrant Crospovidone Type B

| | | 15.0 mg | 15.5 mg |
|---|---|---|---|
| | | tafamidis | tafamidis |
| | | tablet | tablet |
| | | (Example 7) | (Example 8) |
| Ingredient | Function | (mg/tablet) | (mg/tablet) |
| Tafamidis (free acid) | Active | 15.00 | 15.50 |
| | Ingredient | | |
| Microcrystalline | Diluent | 68.69 | 70.96 |
| cellulose | | | |
| Lactose Monohydrate | Diluent | 34.33 | 35.47 |
| Crospovidone Type B | Disintegrant | 5.18 | 5.35 |
| (super fine) ^{a} | | | |
| Magnesium stearate ^{a c} | Lubricant | 0.65 | 0.67 |
| Crospovidone Type B | Disintegrant | 5.18 | 5.35 |
| (super fine) ^{b} | | | |
| Magnesium stearate ^{b c} | Lubricant | 0.32 | 0.33 |
| Total Tablet Core Weight | | 129.35 | 133.63 |
| Opadry ® II yellow | Film-coating | 5.17 | 5.35 |
| | agent | | |
| Opadry ® II white | Film-coating | | |
| | agent | — | — |

| | | |
|---|---|---|
| Total Film-Coated Tablet Weight | 134.52 | 138.98 |

^{a} Intra-granular
^{b} Extra-granular
^{c} non-bovine

Table 1D: Examples 9, 10 and 11-Composition of Tafamidis 59.0 mg, 60.0 mg and 62.0 mg Film-Coated Tablets using 8% (w/w) Disintegrant Crospovidone Type B

TABLE 1D
Examples 9, 10 and 11 - Composition of Tafamidis 59.0 mg, 60.0 mg and 62.0
mg Film-Coated Tablets using 8% (w/w) Disintegrant Crospovidone Type B

| | | 59.0 mg | 60.0 mg | 62.0 mg |
|---|---|---|---|---|
| | | tafamidis | tafamidis | tafamidis |
| | | tablet | tablet | tablet |
| | | (Example 9) | (Example 10) | (Example 11) |
| Ingredient | Function | (mg/tablet) | (mg/tablet) | (mg/tablet) |
| Tafamidis (free | Active | 59.00 | 60.0 | 62.0 |
| acid) | Ingredient | | | |
| Microcrystalline | Diluent | 270.09 | 274.67 | 283.83 |
| cellulose | | | | |
| Lactose | Diluent | 135.02 | 137.31 | 141.89 |
| Monohydrate | | | | |
| Crospovidone | Disintegrant | 20.36 | 20.70 | 21.40 |
| Type B (super | | | | |
| fine) ^{a} | | | | |
| Magnesium | Lubricant | 2.56 | 2.61 | 2.69 |
| stearate ^{a c} | | | | |
| Crospovidone | Disintegrant | 20.36 | 20.70 | 21.40 |
| Type B (super | | | | |
| fine) ^{b} | | | | |
| Magnesium | Lubricant | 1.26 | 1.28 | 1.32 |
| stearate ^{b c} | | | | |
| Total Tablet Core Weight | | 508.65 | 517.27 | 534.53 |
| Opadry ® II | Film-coating | — | — | — |
| yellow | agent | | | |
| Opadry ® II white | Film-coating | 20.35 | 20.69 | 21.38 |
| | agent | | | |

| | | | |
|---|---|---|---|
| Total Film-Coated Tablet Weight | 529.00 | 537.96 | 555.91 |

^{a} Intra-granular
^{b} Extra-granular
^{c} non-bovine

All patents and publications described hereinabove are hereby incorporated by reference in their entirety. While the invention has been described in terms of various preferred embodiments and specific examples, the invention should be understood as not being limited by the foregoing detailed description, but as being defined by the appended claims and their equivalents.

1. A pharmaceutical composition which is a tablet comprising tafamidis free acid, one or more diluents, 7-9% (w/w %) disintegrant and a lubricant.

2. The pharmaceutical composition of claim 1 which is a tablet wherein the disintegrant is crospovidone.

3. The pharmaceutical composition of claim 2 which is a tablet wherein the disintegrant is crospovidone type b.

4. The pharmaceutical composition of claim 3 which is a tablet wherein the one or more diluents are selected from microcrystalline cellulose and lactose monohydrate.

5. The pharmaceutical composition of claim 4 which is a tablet wherein the lubricant is magnesium stearate.

6. The pharmaceutical composition of claim 5 which is a tablet comprising about 8% (w/w %) crospovidone type b.

7. The pharmaceutical composition of claim 6 which is a tablet comprising about 11.6% (w/w %) tafamidis free acid.

8. The pharmaceutical composition of claim 7 which is a tablet comprising 12.2 mg tafamidis free acid.

9. The pharmaceutical composition of claim 7 which is a tablet comprising 61 mg tafamidis free acid.

10. The pharmaceutical composition of claim 8 or claim 9 which is a tablet comprising about 79.65% (w/w %) of one or more diluents which are selected from microcrystalline cellulose and lactose monohydrate.

11. The pharmaceutical composition of claim 10 which is a tablet comprising about 53.1% (w/w %) microcrystalline cellulose and about 26.55% lactose monohydrate.

12. The pharmaceutical composition of claim 11 which is a tablet comprising about 0.75% (w/w %) of magnesium stearate.

13. The pharmaceutical composition of any one of claim 12 wherein the tablet is film coated.

14. The pharmaceutical composition of claim 13 wherein the tablet film coating comprises hydroxypropyl methylcellulose and lactose.

15. The pharmaceutical composition of claim 14 wherein the tablet film coating is about 4% (w/w) of the overall weight of the film coated tablet.

16. A pharmaceutical composition which is a tablet comprising about 11.6% (w/w %) tafamidis free acid; about 53.1% (w/w %) microcrystalline cellulose; about 26.55% (w/w %) lactose monohydrate; about 8.0% (w/w %) crospovidone type b and about 0.75% (w/w %) magnesium stearate.

17. A pharmaceutical composition which is a tablet comprising 11.6%±0.1% (w/w %) tafamidis free acid; 53.1%±0.1% (w/w %) microcrystalline cellulose; 26.55%±0.1% (w/w %) lactose monohydrate; 8.0%±0.1% (w/w %) crospovidone type b and 0.75%±0.1% (w/w %) magnesium stearate.

18. The pharmaceutical composition of claim 16 or 17 wherein the tablet is film coated.

19. The pharmaceutical composition of any one of claim 18 wherein the tablet film coating comprises hydroxypropyl methylcellulose and lactose.

20. The pharmaceutical composition of claim 19 wherein the tablet film coating is 4.0%±0.1% (w/w %) of the overall weight of the film coated tablet.

21. A pharmaceutical composition which is a tablet comprising about 12.20 mg tafamidis free acid, about 55.85 mg microcrystalline cellulose, about 27.92 mg lactose monohydrate, about 8.42 mg crospovidone type b and about 0.79 mg magnesium stearate.

22. The pharmaceutical of claim 21 which is a tablet comprising 12.20 mg tafamidis free acid, 55.85 mg microcrystalline cellulose, 27.92 mg lactose monohydrate, 8.42 mg crospovidone type b and 0.79 mg magnesium stearate.

23. The pharmaceutical composition of claim 22 wherein the tablet is film coated.

24. The pharmaceutical composition of claim 23 wherein tablet film coating comprises hydroxypropyl methylcellulose and lactose.

25. The pharmaceutical composition of claim 24 wherein the tablet film coating weighs 4.21 mg.

26. A pharmaceutical composition which is a tablet comprising about 61.00 mg tafamidis free acid, about 279.25 mg microcrystalline cellulose, about 139.6 mg lactose monohydrate, about 42.10 mg crospovidone type b and about 2.95 mg magnesium stearate.

27. The pharmaceutical composition of claim 26 which is a tablet comprising 61.00 mg tafamidis free acid, 279.25 mg microcrystalline cellulose, 139.6 mg lactose monohydrate, 42.10 mg crospovidone type b and 2.95 mg magnesium stearate.

28. The pharmaceutical composition of claim 27 wherein the tablet is film coated.

29. The pharmaceutical composition of claim 28 wherein the tablet film coating comprises hydroxypropyl methylcellulose and lactose.

30. The pharmaceutical composition of claim 29 wherein the tablet film coating weighs 21.05 mg.

31. The pharmaceutical composition of claim 7 which is a tablet comprising about 13.0 mg, about 13.5 mg, about 14.0 mg, about 14.5 mg, about 15.0 mg, about 15.5 mg, about 59.0 mg, about 60.0 mg or about 62 mg tafamidis free acid.

32. The pharmaceutical composition of claim 31 which is a tablet comprising 13.0 mg, 13.5 mg, 14.0 mg, 14.5 mg, 15.0 mg, 15.5 mg, 59.0 mg, 60.0 mg or 62.0 mg of tafamidis free acid.

33. The pharmaceutical composition of claim 32 which is a tablet comprising 13.0 mg tafamidis free acid.

34. The pharmaceutical composition of claim 33 which is a tablet comprising 13.0 mg tafamidis free acid, 59.51 mg microcrystalline cellulose, 29.75 mg lactose monohydrate, 8.98 mg crospovidone type b and 0.85 mg magnesium stearate.

35. The pharmaceutical composition of claim 32 which is a tablet comprising 13.5 mg tafamidis free acid.

36. The pharmaceutical composition of claim 35 which is a tablet comprising 13.5 mg tafamidis free acid, 61.80 mg microcrystalline cellulose, 30.89 mg lactose monohydrate, 9.32 mg crospovidone type b and 0.88 mg magnesium stearate.

37. The pharmaceutical composition of claim 32 which is a tablet comprising 14.0 mg tafamidis free acid.

38. The pharmaceutical composition of claim 37 which is a tablet comprising 14.0 mg tafamidis free acid, 64.09 mg microcrystalline cellulose, 32.04 mg lactose monohydrate, 9.66 mg crospovidone type b and 0.91 mg magnesium stearate.

39. The pharmaceutical composition of claim 32 which is a tablet comprising 14.5 mg tafamidis free acid.

40. The pharmaceutical composition of claim 39 which is a tablet comprising 14.5 mg tafamidis free acid, 66.38 mg microcrystalline cellulose, 33.18 mg lactose monohydrate, 10.00 mg crospovidone type b and 0.94 mg magnesium stearate.

41. The pharmaceutical composition of claim 32 which is a tablet comprising 15.0 mg tafamidis free acid.

42. The pharmaceutical composition of claim 41 which is a tablet comprising 15.0 mg tafamidis free acid, 68.69 mg microcrystalline cellulose, 34.33 mg lactose monohydrate, 10.36 mg crospovidone type b and 0.97 mg magnesium stearate.

43. The pharmaceutical composition of claim 32 which is a tablet comprising 15.5 mg tafamidis free acid.

44. The pharmaceutical composition of claim 43 which is a tablet comprising 15.5 mg tafamidis free acid, 70.96 mg microcrystalline cellulose, 35.47 mg lactose monohydrate, 10.70 mg crospovidone type b and 1.00 mg magnesium stearate.

45. The pharmaceutical composition of claim 32 which is a tablet comprising 59.0 mg tafamidis free acid.

46. The pharmaceutical composition of claim 45 which is a tablet comprising 59.0 mg tafamidis free acid, 270.09 mg microcrystalline cellulose, 135.02 mg lactose monohydrate, 40.72 mg crospovidone type b and 3.82 mg magnesium stearate.

47. The pharmaceutical composition of claim 32 which is a tablet comprising 60.0 mg tafamidis free acid.

48. The pharmaceutical composition of claim 47 which is a tablet comprising 60.0 mg tafamidis free acid, 274.67 mg microcrystalline cellulose, 137.31 mg lactose monohydrate, 41.40 mg crospovidone type b and 3.89 mg magnesium stearate.

49. The pharmaceutical composition of claim 32 which is a tablet comprising 62.0 mg tafamidis free acid.

50. The pharmaceutical composition of claim 49 which is a tablet comprising 62.0 mg tafamidis free acid, 283.83 mg microcrystalline cellulose, 141.89 mg lactose monohydrate, 42.80 mg crospovidone type b and 4.01 mg magnesium stearate.

51. The pharmaceutical composition of any one of claims 31 to 50 wherein the tablet is film coated.

52. The pharmaceutical composition of claim 51 wherein the tablet film coating comprises hydroxypropyl methylcellulose and lactose.

53. The pharmaceutical composition of claim 52 wherein the tablet film coating is about 4% (w/w) of the overall weight of the film coated tablet.

54. A method of treating transthyretin amyloidosis in a patient comprising administering a pharmaceutical composition according to any one of claims 1 to 53 to the patient.

55. The method of claim 54 wherein the transthyretin amyloidosis is transthyretin cardiomyopathy (ATTR-CM) or transthyretin polyneuropathy (ATTR-PN).

56. Use of a pharmaceutical composition according to any one of claims 1 to 53 for treatment of transthyretin amyloidosis.

57. The use according to claim 56 wherein the transthyretin amyloidosis is transthyretin cardiomyopathy (ATTR-CM) or transthyretin polyneuropathy (ATTR-PN).
